(19) United States
(12) Patent Application Publication
(10) Pub. No.: US 2012/0010601 A1
(43) Pub. Date: Jan. 12, 2012

(54) ORTHOPEDIC CEMENT AND USE OF SAME IN RADIATION THERAPY

(75) Inventors: Scott D. Simon, Richmond, VA (US); George Ding, Franklin, TN (US); Joseph Cheng, Nashville, TN (US)

(73) Assignee: VANDERBILT UNIVERSITY, Nashville, TN (US)

(21) Appl. No.: 13/149,068

(22) Filed: May 31, 2011

Related U.S. Application Data
(60) Provisional application No. 61/363,035, filed on Jul. 9, 2010.

(51) Int. Cl. A61B 17/00; A61P 35/04; A61K 51/06

(52) U.S. Cl. 606/1; 424/1.11

(57) ABSTRACT

A method of treating diseased tissue in a patient, the diseased tissue being proximate a hardened previously implanted bone cement including relatively high atomic number elements in a patient. The method includes generating a photon beam and directing the generated photon beam into the patient in a direction such that at least a portion of the photon beam impinges on the hardened bone cement and generates Compton interaction knock-out electrons from the high atomic number elements included in the hardened bone cement as a result of interaction of the at least a portion of the photon beam with the bone cement, wherein the direction of the photon beam is such that the at least a portion of the photon beam impinges on the hardened bone cement so that at least some of the Compton interaction knock-out electrons impinge upon the diseased tissue.

### CROSS-REFERENCE TO RELATED APPLICATIONS

This application claims priority to U.S. Provisional patent application No. 61/363,035, filed Jul. 9, 2010. The contents of this application is hereby incorporated by reference herein in its entirety.

### BACKGROUND

1. Field Of The Invention

The present invention relates generally to orthopedic cement, and more specifically, to using an orthopedic cement in radiotherapy.

2. Related Art

Certain conditions, defects, deformities and injuries may lead to structural instabilities in a patient's bone, cartilage or other connective tissue. Such structural instability is particularly problematic in a patient's spinal column due to the potential for nerve or spinal cord damage pain and other manifestations. Such structural instability may occur as a result of pathologic fractures caused by, for example, a tumor within a vertebral body or the like.

FIG. 1 is a perspective view of a segment 100 of a human spinal column. An individual's spinal column (also referred to as the vertebral column) extends from the person's skull (not shown) to the pelvis (also not shown) and consists of 33 individual bones known as vertebrae 102. Two such vertebrae 102 are illustrated in FIG. 1. Between each vertebra 102 is a soft, gel-like cushion known as an intervertebral disc 104 which absorbs pressure and prevents vertebrae 102 from contacting each other. There are two such intervertebral discs 104 illustrated in FIG. 1. Each vertebra 102 is held to other vertebrae in the spinal column by ligaments (not shown) which also connect to vertebrae 102 to the individual's muscles. Additional tendons (not shown) also fasten muscles to vertebrae 102.

Each vertebra 102 comprises a centrum or vertebral body 106 comprised of dense cortical bone forming the anterior portion of vertebra 102. Vertebral bodies 106 collectively provide structural support to the spinal column. Posterially extending from vertebral body 106 is a spinous process 122 and two transverse processes 120 on opposing lateral sides of spinous process 122. The portion of vertebra 102 which extends between transverse processes 120 and which is disposed between transverse processes 120 and vertebral body 106 is referred to as pedicle 118. Processes 120, 122 add structural rigidity, assist in articulation of vertebrae 102 in conjunction with the individual's ribs (not shown), and serve as muscle attachment points.

Each vertebra 102 further comprises lamina 110 which form the walls of spinal canal 112. Extending through spinal canal 112 is spinal cord 114.

Damage and structural instability to a patient's spine may occur in a variety of circumstances. One notable cause of structural instability in an individual's spinal column is due to bone metastases associated with the advancement of cancer cells originating at other locations in the individual's body, where a tumor develops within a vertebral body. Spinal metastasis occurs in 5-10% of all patients who suffer from cancer. Barron, K. D. et al., Neurology 9:91-106 (1959). Furthermore, autopsy studies have found metastatic involvement of the spinal column in 90% of patients with prostate cancer, in 75% of patients with breast cancer, 45% of patients with lung carcinoma, 55% of patients with melanoma, and 30% of patients with renal carcinoma. Lenz, M. et al., Ann Surg 93:278-293 (1931); Sundaresan N, et al., Tumors of the Spine: Diagnosis and Clinical Management. Philadelphia: W B Saunders: pp 279-304 (1990); Wong, D.A. et al., Spine, 15:1-4 (1990).

About 10% of patients who suffer from spinal metastasis will subsequently develop spinal cord compression. Schaberg J. et al., Spine 10:19-20 (1985); Sundaresan N, et al., Neurosurgery, 29:645-650 (1991). The metastatic spinal lesions affect vertebral body 106 and pedicle 118 in approximately 85% of the patients suffering from spinal metastasis. Riaz et al., supra. The distribution of the metastatic lesions according to the level of vertebrae in various spinal segments is: thoracic spine 70%, lumbar spine 20% and cervical spine 10%. Barron et al., supra; Gilbert R W, et al., Ann Neurol, 3:40-51 (1978). Typically, the posterior region of vertebral body 106 is invaded first, with the anterior region, lamina 110, and pedicles 118 invaded at a later time. Adams M, et al., Contemp Neurosurg, 23:1-5 (2001).

The treatment of spinal metastasis is primarily palliative except in rare circumstances. Available treatments include chemotherapy, radiotherapy (also referred to as radiosurgery and radiation therapy), hormonal therapy and/or surgery. Radiotherapy has proven successful for treatment of spinal metastasis. Radiotherapy is recommended when surgery is not possible or considered too risky.

Kyphoplasty and vertebroplasty involve the percutaneous transpedicular injection of an orthopedic or bone cement into the compressed vertebral body 106 to “decompress” the compressed vertebral body, thus restoring at least some of body height and, also reducing pain. Specifically, in kyphoplasty, a needle is introduced into the compressed vertebral body. A small tube is slid over the needle. Through this tube, a balloon tipped catheter is inserted into the compressed vertebral body. The balloon is slowly inflated, raising the compressed vertebral body to its normal height. The balloon creates a space in the vertebral body as it inflates. This space allows for bone cement to be placed in the space under a low pressure. This substantially reduces the risk of cement leaking out of the vertebral body. When the cement hardens, the cement supports the vertebral body at its normal height.

Vertebroplasty also uses bone cement to support the vertebral body at its normal height. However, unlike in kyphoplasty, in vertebroplasty, cement is utilized to raise the compressed vertebral body to its normal height. That is, no balloon is utilized to raise the compressed vertebral body to its normal height.

### SUMMARY

In one aspect of the present invention, there is a method of treating diseased tissue in a patient, the diseased tissue being proximate a hardened previously implanted bone cement including relatively high atomic number elements in a patient. The method comprises generating a photon beam, directing the generated photon beam into the patient in a direction such that the photon beam impinges on the hardened bone cement and generates Compton interaction knock-out electrons from the high atomic number elements included in the hardened bone cement as a result of interaction of the photon beam with the bone cement, wherein the direction of the photon beam is such that the photon beam impinges on the hardened bone cement so that at least some of the Compton interaction knock-out electrons impinge upon the diseased tissue.

According to yet another aspect of the present invention, there is a composition for bone cement used in at least one of kyphoplasty and vertebroplasty consisting essentially of relatively high atomic number elements at about 20% to 40% by weight prior to hardening of the bone cement, and polymethylmethacrylate.

According to yet another aspect of the present invention, there is a method of treating spinal metastasis in a patient, the spinal metastasis including a tumor proximate a hardened cement in a vertebral body of a spinal body of a patient. The method includes developing a treatment regime for treating the spinal metastasis by computationally estimating with an electronic computer a secondary radiation dose to be received by the tumor resulting from Compton interaction knock-out electrons generated from the hardened cement as a result of the impingement of a photon beam on the hardened cement in a first direction. The method further includes directing a photon beam to impinge on the hardened cement to generate the Compton interaction knock-out electrons based on the developed treatment regime so as to provide a secondary radiation dose to the tumor.

### BRIEF DESCRIPTION OF THE DRAWINGS

Illustrative embodiments of the present invention are described herein with reference to the accompanying drawings, in which:

FIG. 1 is a perspective view of a segment of a human spinal column;

FIG. 2 is a side view of a segment of a human spinal column including a pathologic fracture;

FIG. 3a is a side view of a segment of a human spinal column depicting a needle inserted in vertebral body during a kyphoplasty procedure;

FIG. 3b is a side view of a segment of a human spinal column depicting the step of inserting a balloon during a kyphoplasty procedure;

FIG. 4 is a side view of a segment of a human spinal column depicting the step of inflating a balloon during a kyphoplasty procedure;

FIG. 5 is a side view of a segment of a human spinal column depicting injection of bone cement into a balloon during a kyphoplasty procedure;

FIG. 6 is a side view of a segment of a human spinal column depicting the results of a kyphoplasty procedure;

FIG. 7 is a side view depicting radiotherapy treatment according to an embodiment of the present invention;

FIG. 8 is a top view of a section of a vertebral body including hardened bone cement and a tumor treated by radiotherapy according to an embodiment of the present invention;

FIG. 9 depicts a graph of radiation dose a function of distance according to an embodiment of the present invention;

FIG. 10 depicts another graph of radiation dose a function of distance according to an embodiment of the present invention;

FIG. 11 is a flow chart presenting steps of a method according to an embodiment of the present invention; and

FIG. 12 is a flow chart presenting steps associated with developing a radiation therapy regime for a patient.

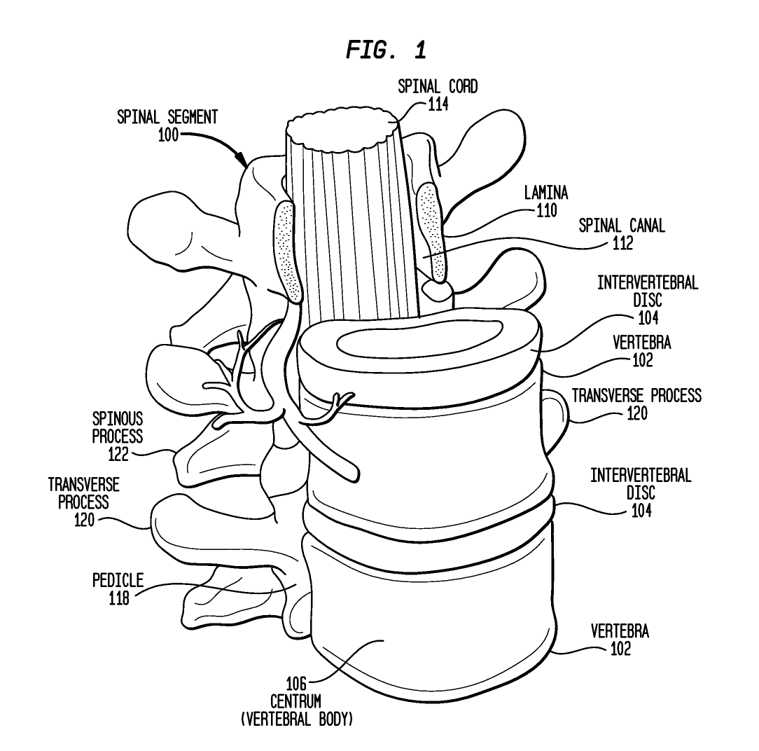

### DETAILED DESCRIPTION

Aspects of the present invention are generally directed to a bone cement for use in radiotherapy (also referred to as radiotherapy) that generates Compton interaction knock-out electrons when the bone cement is exposed to radiation, thereby increasing the energy that may be delivered to a target tissue proximate the bone cement. In some embodiments, the target tissue is a diseased tissue, such as a tumor, and the bone cement is implanted in a vertebral body of a spinal body of a patient. While embodiments of the present invention have been described herein in terms of the target tissue being a tumor, and the bone cement being implanted in a spinal body, in other embodiments, the target tissue may be other types of diseased tissue, and the bone cement may be implanted in other locations within the patient.

In one aspect of the present invention, there is a method of treating a target tissue that is a diseased tissue, such as a spinal metastasis in a patient, the spinal metastasis including diseased tissue, such as a tumor, proximate a hardened bone cement including high atomic number elements in a vertebral body of a spinal body of a patient. The method includes generating a photon beam, directing the generated photon beam into the patient in a direction such that the photon beam impinges on the hardened bone cement, and generating Compton interaction knock-out electrons from the hardened bone cement. In the method, the direction of the photon beam is such that the photon beam impinges on the hardened bone cement so that at least some of the Compton interaction knock-out electrons impinge upon the tumor.

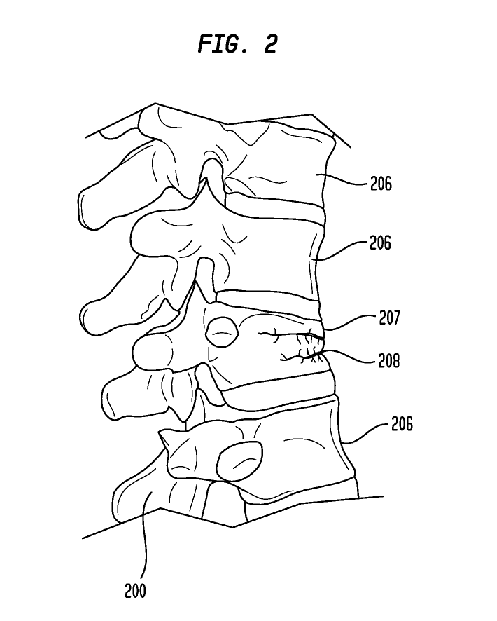

FIG. 2 is a side view of a segment 200 of a spinal column of a human patient. Segment 200 includes a plurality of healthy vertebral bodies 206 and a compressed vertebral body 207 having a pathologic fracture 208. The compressed vertebral body 207 has collapsed to a compressed dimension as a result of a tumor within the vertebral body (not shown).

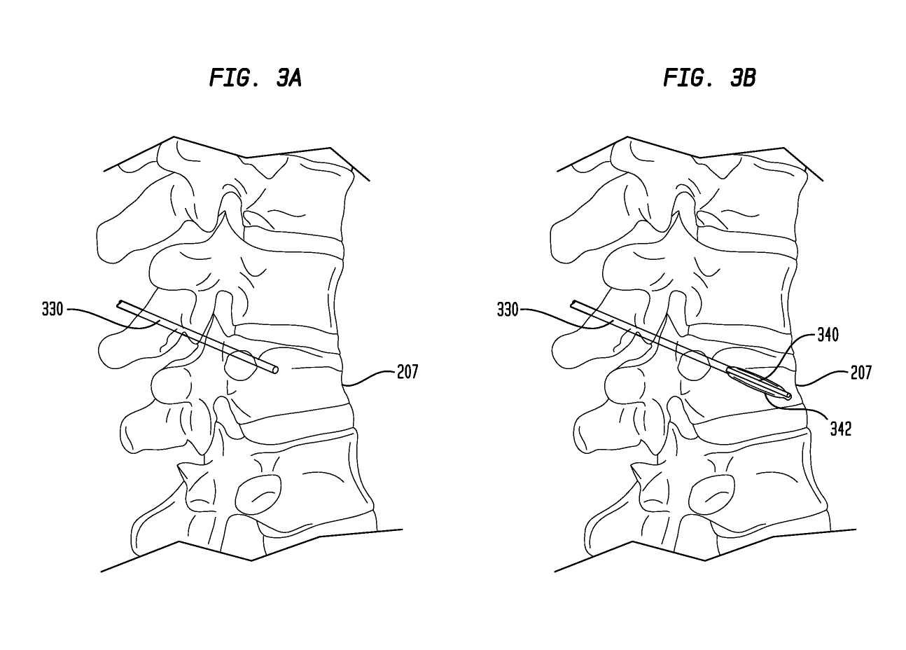

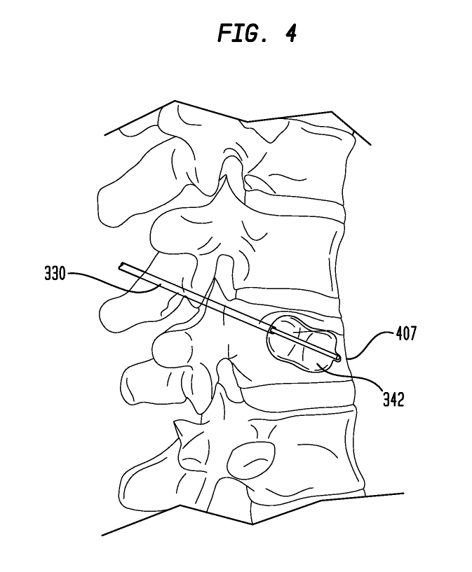

Certain embodiment(s) of the present invention include(s) combining kyphoplasty with radiotherapy to treat the diseased vertebral body 207. First, kyphoplasty is implemented on the patient to return, partially or fully, the compressed vertebral body 207 to its original height. One specific embodiment is shown in FIG. 3a. A needle 330 is percutaneously inserted through a patient's back so that the tip of the needle reaches the interior of the compressed vertebral body 207. As may be seen in FIG. 3b, a balloon catheter 340 including a balloon 342 is slid over the needle 330 into the compressed vertebral body 207. A gas, such as air or nitrogen or another inert gas, etc., is directed through the catheter 340 into the balloon 342, thus inflating the balloon 342 so that it has a volume as shown by way of example in FIG. 4. Inflation of balloon 342 returns vertebral body 207, fully or partially, to its original height, thus resulting in an decompressed vertebral body 407 (see FIG. 4).

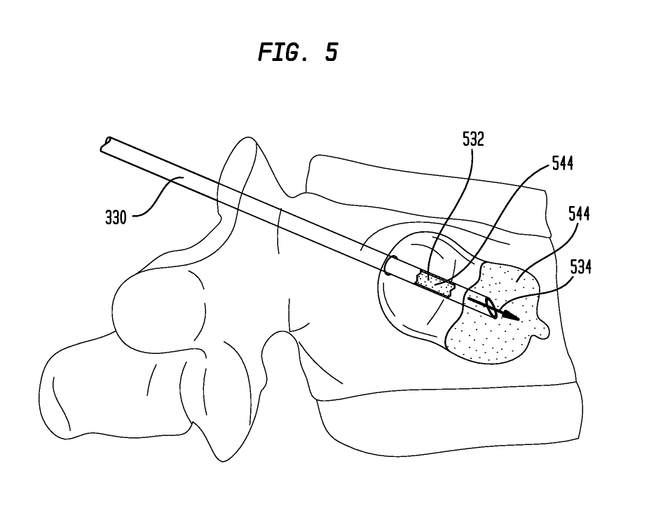

Next, referring to FIG. 5, viscous bone cement 544 is directed through lumen 532 of needle 330 in the direction of arrow 534. Bone cement 544 flows or is otherwise dispersed into balloon 342 and displaces the gas in the balloon as it fills the balloon. Next, the viscous bone cement 544 is permitted to harden via, for example, a chemical reaction that hardens the bone cement relatively quickly, or via another type of reaction.

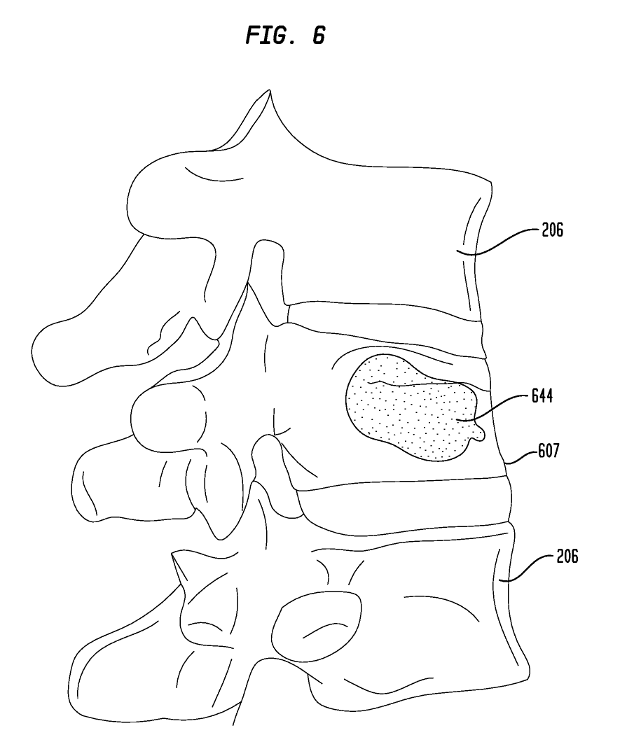

FIG. 6 depicts hardened bone cement 644 within decompressed vertebral body 407. Hardened bone cement 644 serves as a load bearing member that is capable of bearing the load placed onto hardened bone cement 644, thereby retaining the structural integrity necessary to prevent recompression of decompressed vertebral body 407. This permits decompressed vertebral body 407 to sufficiently support the weight imposed on it by the spinal column. Accordingly, the spinal column of FIG. 6 includes a decompressed vertebral body 607 including hardened bone cement 644.

In an embodiment, fluoroscopic or other imaging may be used to ensure that the viscous bone cement 544/hardened bone cement 644 is properly positioned.

After the viscous bone cement 544 is injected into balloon 344, needle 330 and catheter 340 are removed. At this point, the kyphoplasty portion of the procedure is completed.

In the present invention, the viscous bone cement 544, and thus the hardened bone cement 644, utilized in the kyphoplasty procedure detailed above contains a material that ejects electrons when exposed to high frequency radiation, such as X-rays used in radiotherapy. More specific features of the material will be discussed in greater detail below.

As noted above, according to the present invention, at least some of the ejected electrons impinge upon a tumor that has grown in and/or adjacent to compressed vertebral body 207 and remains present in and/or adjacent to decompressed vertebral body 607. Typically, this tumor is what initiated the pathologic fracture 208 that resulted in the compression of compressed vertebral body 207. According to the present invention, the ejected electrons that impinge upon the tumor may aid in partially stunting the growth of the tumor, and in many instances, reverse the growth of the tumor (e.g., the electrons are used for curative or adjuvant treatment). In some instances, the electrons instead simply reduce the severity of the symptoms associated with the presence of the tumor in decompressed vertebral body 607 (e.g., the electrons are used for palliative treatment). Hereinafter, stunting the growth of the tumor, reversing the growth of the tumor and reducing the severity of the symptoms associated with the presence of the tumor (curative, adjuvant and palliative, etc., treatment) are collectively referred to as “treating the tumor.”

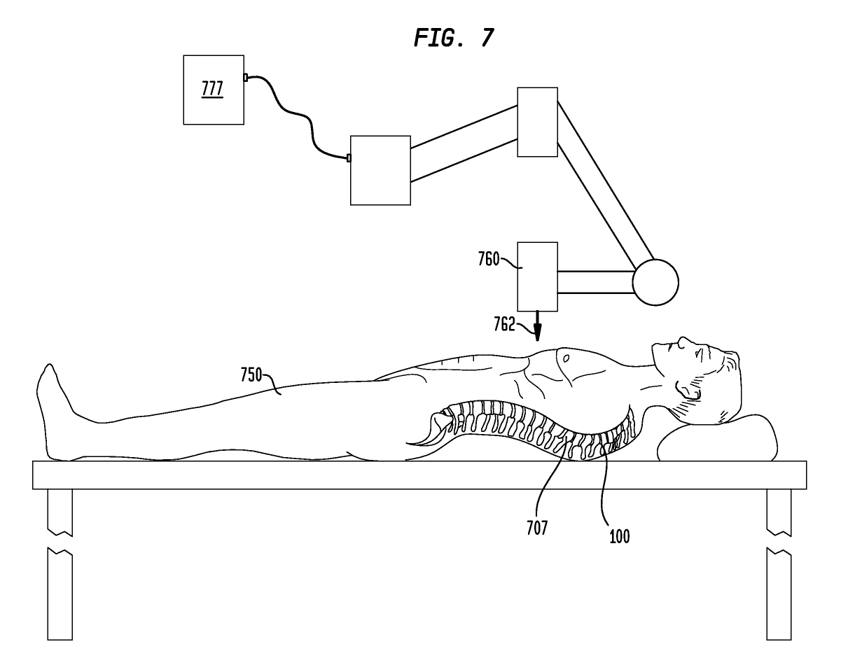

Specifically, referring to FIG. 7, a patient 750 is depicted as having a spinal segment 100 including decompressed vertebral body 706b in which hardened bone cement 644 and a tumor (not shown) is located. According to the present invention, patient 750 is given radiotherapy to treat the tumor. In FIG. 7, an external photon beam generator 760 such as a linear accelerator (LINAC), or a gamma knife is used to generate a photon beam 762 in the form of high-energy X-rays that originates external to the patient 750. This beam is directed towards the decompressed vertebral body 706b so that in impinges upon the tumor and/or hardened bone cement in decompressed vertebral body 706b to treat the tumor. In an exemplary embodiment, the photon beam 762 generated by the external photon beam generator 760 is a beam of 6 MV photons, although in other embodiments, the energy level of the beam may be higher or lower than 6 MV. Any energy level of the photon beam 762 that may permit the present invention to be practice may be used in embodiments of the present invention. In some embodiments, any stereotactic radiation source may be used.

In practice, photon beam generator 760 is moved about patient 750 so that multiple photon beams 762 impinge on the tumor and/or the hardened bone cement in decompressed vertebral body 706b from different directions. The spatial intersection of these multiple photon beams 762 (albeit at different temporal locations) results in a collective dose of primary radiation received by the tumor. Herein, the dose of radiation measured at a given location solely as a result of a single photon beam 762 impinging upon the tumor is referred to as a primary one beam radiation dose, and the collective dose of radiation measured at a given location solely as a result of the “intersection” of multiple photon beams 762 impinging upon the tumor is referred to as a primary collective radiation dose.

As noted above, multiple photon beams 762 impinge on the tumor from different directions, thus treating the tumor. However, at least portions of some of the photon beams 762 impinge on the hardened bone cement in the decompressed vertebral body 706b, either by completely bypassing the tumor or after passing through the tumor. Embodiments of the present invention harnesses the portions of the photon beam 762 that impinge on the hardened bone cement 644 to enhance the treatment of the tumor, as will now be described.

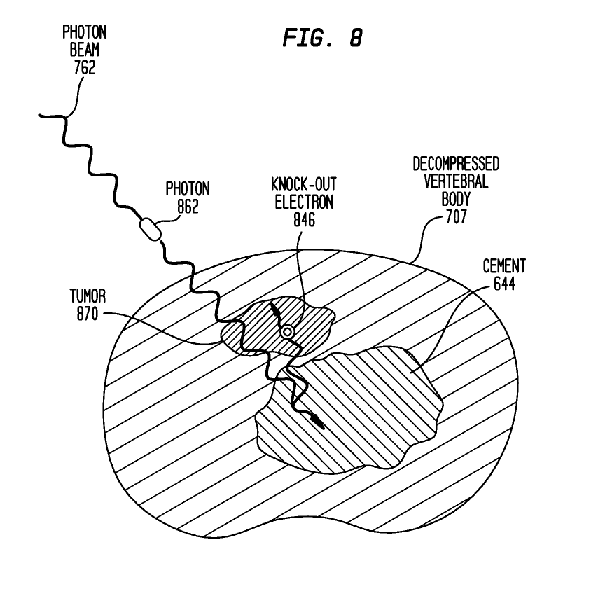

FIG. 8 depicts a cross-sectional view of a decompressed vertebral body 706b taken along the longitudinal direction of the spinal column. Referring to FIG. 8, a photon 862 of photon beam 762 passes through a portion of decompressed vertebral body 706b and tumor 870 (thus providing a primary one beam radiation dose to the tumor 870), to impinge upon hardened bone cement 644. Photon beam 762 impinging on hardened bone cement 644, whether or not after passing through tumor 870, is sufficient to result in the ejection of a “knock-out” electron 846 from hardened bone cement 644. The ejection of “knock-out” electron 846 is a result of Compton interaction with material of hardened bone cement 846. In some instances, electron 864 travels towards tumor 870. It is noted that in some instances, tumor 870 may be fully or partially located in decompressed vertebral body 706, or may be external to vertebral body 706 proximate the vertebral body.

At least some of the energy of electron 846 is dissipated in tumor 870, thus imparting a secondary one beam radiation dose to the tumor. The collection of this secondary radiation dose from the intersection of the beams results in the secondary collective radiation dose. This results in a total one beam radiation dose received by tumor 870 that is greater than the primary one beam radiation dose received by the tumor, and in some instances, equals the sum of the primary and secondary.

In some embodiments, multiple photon beams 762 are directed toward decompressed vertebral body 706b and/or tumor 870 at different angles of incidence relative to one another. This increases in the dose of radiation received by tumor 870 thereby enhancing treatment of the tumor. In an embodiment, fewer photon beams 762 may be needed and/or the energy of some or all of the photon beams 762 may be reduced to achieve the same results.

In an embodiment, photon 862 may pass through tumor 870, and may dissipate some of its energy into the tumor, before impinging upon hardened bone cement 644. The interaction of this photon 862 with bone cement 644 may still result in a knock-out electron 864 as detailed above, even though some of the photon's energy has been dissipated into tumor 870.

In view of the above, the present invention includes treating tumor 870 by exposing the tumor to primary radiation in the form of photon 862 from photon beam 762, and treating the tumor 870 by exposing the tumor 870 to secondary radiation in the form of knock-out electron 846 from hardened bone cement 644.

Still referring to FIG. 8, in an embodiment, there is an increase to the total one beam radiation dose (primary plus secondary) immediately proximal to hardened bone cement 644 and/or a decrease in the collective radiation dose behind the bone cement. In an embodiment, depending on the materials used to form the hardened bone cement 644, the increase in the total one beam radiation dose immediately proximal to the bone cement at the location at which photon beam 862 impinges upon the bone cement may be as much as about 20% or more, this increase being at least in part due to the Compton interaction knock-out electrons 846. Thus, if tumor 870 is proximate that location, the total one beam radiation dose received by the tumor will be increased by as much as about 20% as a result of the addition of the secondary one beam radiation dose to the primary one beam radiation dose. Also depending on the makeup of the hardened bone cement 644 and/or the distance photon 862 travels through the hardened bone cement 644, the decrease in the total one beam radiation dose behind the bone cement may be about 1-2% to as great as about 10% or more. Accordingly, bone cement 644 provides a shielding effect from the energy of photon beam 762 with respect to the tissue behind the bone cement, such as, for example, the spinal canal 112 (and/or spinal cord and/or nerve roots).

In an embodiment, the increase in the total collective radiation dose immediately proximal hardened bone cement 644 and the decrease in the total collective radiation dose behind the bone cement may be a function of how the respective directions of the multiple photon beams 762 intersect over the course of the radiotherapy procedure. The intersection of the multiple photon beams 762 is typically unique for each patient 750. In this regard, because an embodiment of the present invention includes tailoring the radiotherapy to a given patient by directing photon beams 762 towards the compressed vertebral body 706b at different directions relative to the patient's spinal segment 100, the total collective radiation doses at various locations may differ.

As may be inferred from the above, the bone cement includes material that may be used as a source of the Compton interaction knock-out electrons. In an exemplary embodiment, the bone cement includes a concentration of a heavy metal which serves as a source of the knock-out electrons. One such heavy mental is tantalum. In an exemplary embodiment, the viscous bone cement 544 includes tantalum at about 40% by weight, substantially evenly disbursed within a mixture of polymethylmethacrylate.

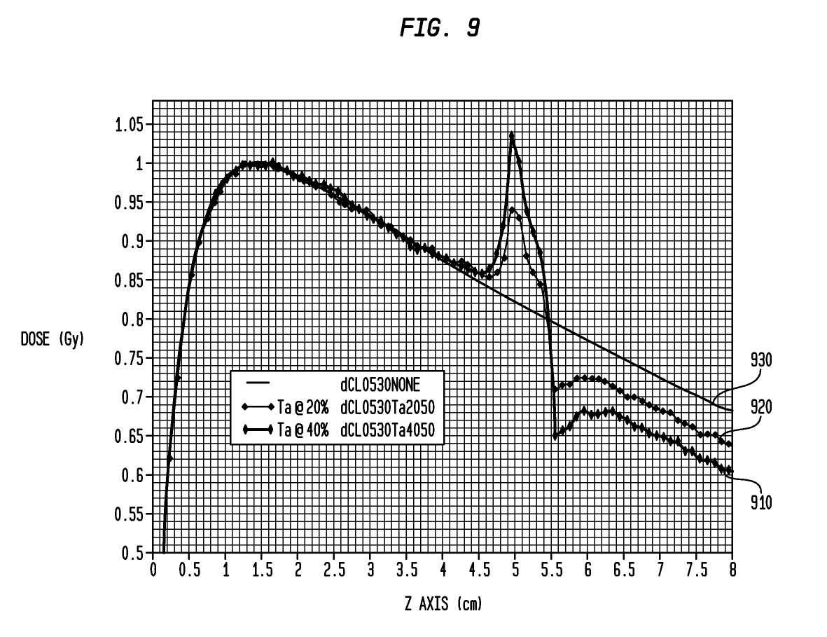

FIG. 9 is a graph depicting how a one beam radiation dose changes when traveling through hardened bone cement made from 40% tantalum by weight (curve 910) and made from 20% tantalum by weight (curve 920), the remainder of the bone cement being PMMA and/or other low density materials (the percentages are prior to hardening). Specifically, FIG. 9 depicts the one beam radiation dose of a photon beam as it passes through water (0 cm to 5 cm and 5.5 cm to 8 cm on the Z axis) and as it passes through and embodiment of the hardened bone cement having the tantalum (5.0 cm to 5.5 cm on the Z axis), where water is representative of body tissue. In FIG. 9, curve 930 represents a control curve of the dose without hardened bone cement (i.e., water from 0 cm to 8 cm on the Z axis).

As may be seen from FIG. 9, the one beam radiation dose spikes at the surface of the hardened bone cement at about 5.0 on the Z axis. Some of the increase is a result of so-called “back-scattering” of the photon beam as it impinges upon the denser material of the hardened bone cement. However, some of the increase is a result of the Compton interaction knock-out electrons. Accordingly, FIG. 9 fairly depicts the increase in one beam radiation dose resulting from the Compton interaction knock-out electrons.

FIG. 9 further details how the one beam radiation dose behind the hardened bone cement is lower than what would otherwise be the case if the tantalum-doped bone cement was not present, as the dose for curves 910 and 920 are lower than curve 930, from 5.5 cm to 8 cm on the Z axis (the area “behind” the hardened bone cement is at 5.0 to 5.5 cm on the Z axis). It is noted that the relatively sudden decrease, and subsequent sudden increase, in one beam dose as the photon beam exits the hardened bone cement is believed to be an artifact of the charged-particle equilibrium that occurs when a beam passes between media of different density. Thus, FIG. 9 depicts a decrease in one beam radiation dose that may be greater than that which actually occurs. Still, overall, if the sudden decrease and increase in the area behind the bone cement is smoothed or otherwise accounted for, FIG. 9 fairly depicts the decrease in one beam radiation dose resulting from the beam passing through the hardened bone cement.

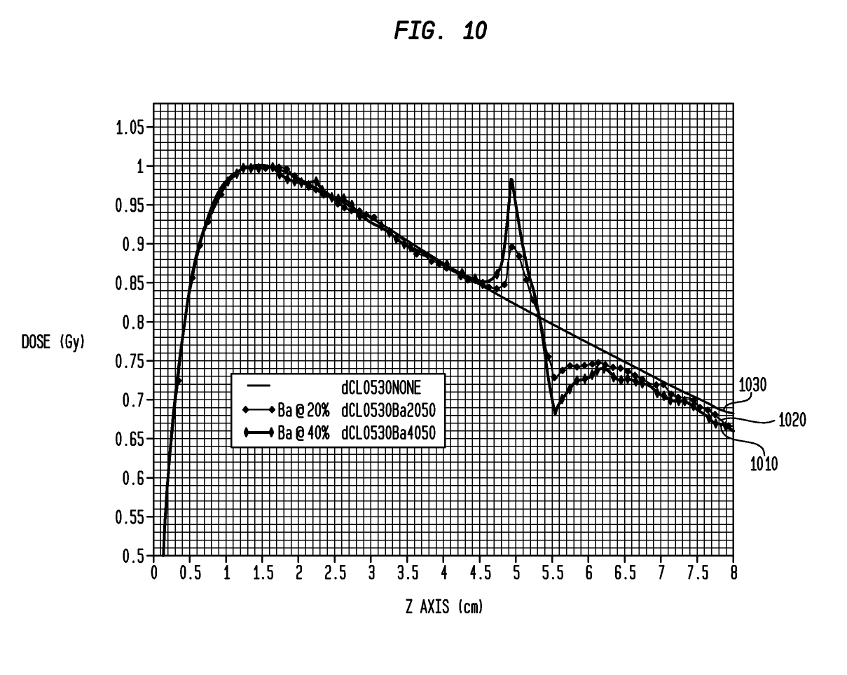

In another embodiment, the bone cement includes a concentration of barium sulfate at about 40% by weight (prior to hardening). FIG. 10 presents a graph depicting how a one beam radiation dose changes when traveling through hardened bone cement made from 40% barium sulfate by weight prior to hardening (curve 1010) and made from 20% barium sulfate by weight prior to hardening (curve 1020), the remainder of the bone cement being PMMA and/or other low density materials. As with FIG. 9, in FIG. 10, curve 1030 represents a control curve of the same dose without hardened bone cement. As may be seen, the effects resulting from the use of tantalum in the bone cement are more significant than the effects resulting from the use of barium sulfate in the bone cement.

Any concentration of elements and/or compounds that will permit knock-out electrons to be ejected from the bone cement to treat a tumor while also providing a bone cement that has sufficient efficacy for use in kyphoplasty may be used in alternative embodiments of the present invention. In this regard, the elements and/or compounds added to the bone cement to increase the one beam radiation dose have a relatively high atomic number. The higher the atomic number of the elements that are used as a source of the knock-out electrons, the greater the potential for the bone cement to increase the collective radiation dose immediately adjacent the bone cement. Also, the higher the atomic number of the elements used as the source of the knock-out electrons, the higher the attenuation distal to the bone cement, thus increasing the shielding effect of the bone cement to tissue behind the bone cement.

Further, the higher the atomic number of the elements used as a source of the knock-out electrons, the lower the quantity of those elements need be contained in the bone cement to obtain a desired increase in the collective radiation dose immediately proximate the bone cement. For example, as shown in FIGS. 9 and 10, an embodiment of the present invention may utilize an amount of tantalum that is less than an amount of barium to obtain an equivalent efficacy. Accordingly, using elements having a higher atomic number lessens the effect of the elements on the biomedical properties of the PMMA because less of the doping substance need be used, thus permitting more PMMA to be included in the bone cement

An embodiment of the presenting invention includes any composition that may be used as an orthopedic bone cement used in kyphoplasty and/or vertebroplasty that also generates sufficient Compton interaction knock-out electrons when exposed to a photon beam. Accordingly, an embodiment of the present invention includes a PMMA bone cement doped with relatively high atomic number elements such as tantalum in amounts that permit the PMMA bone cement to retain its utility as a bone cement for kyphoplasty and/or vertebroplasty.

In an embodiment, the elements used as a source of the knock-out electrons are sufficiently radio opaque and present in sufficient quantities to permit the initial kyphoplasty to be performed under fluoroscopy. Also, follow-up fluoroscopy may be implemented on the patient temporally proximate to one or more radiotherapy sessions.

While the above embodiments are directed towards treating spinal metastases associated with a tumor in a vertebral body, other embodiments of the present invention may be utilized to treat other ailments. In an exemplary embodiment of the present invention, any type of substance that will eject knock-out electrons when exposed to a radiation source as detailed herein may be used to treat any type of diseased tissue.

It is noted that while an embodiment of the present invention has been described in terms of treating spinal metastasis, an exemplary embodiment of the invention includes utilizing the techniques described herein to treat primary spinal tumors, and other types of tumors, as will be readily understood.

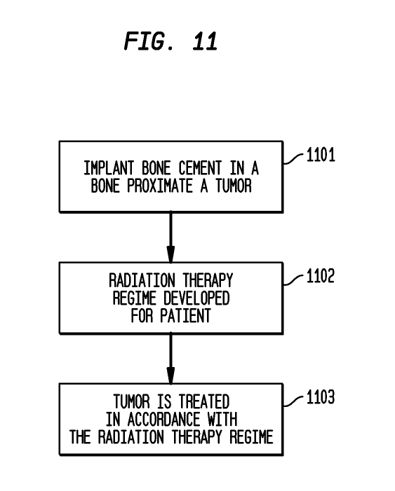

An embodiment of the present invention includes a method of treating a tumor by combining kyphoplasty or vertebroplasty and radiotherapy and relying on the use of Compton interaction knock-out electrons from the hardened bone cement as disclosed herein. Referring to FIG. 11, in a first step 1101, a patient with a tumor or previously having a tumor in or proximate a compressed vertebral body undergoes kyphoplasty (or vertebroplasty) to decompress the compressed vertebral body. In a second step 1102, a radiotherapy regime is developed, specifically tailored to an individual patient, to treat a tumor proximate the hardened bone cement. In a third step 1103, the tumor is treated in accordance with the radiotherapy regime developed in step 1102.

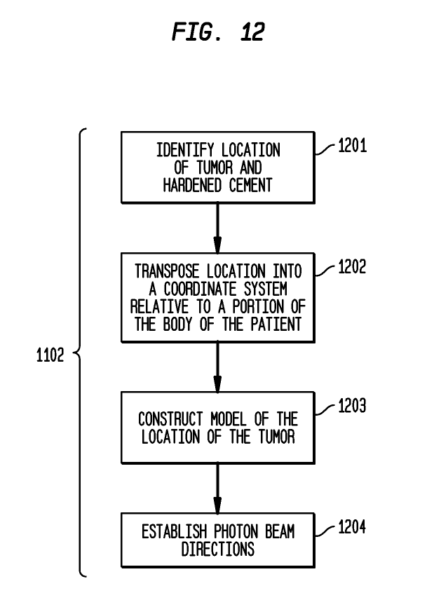

Referring to FIG. 12, step 1102 may include one or more sub-steps, such as step 1201, which entails identifying the location of the tumor and the hardened bone cement in the patient. In an embodiment, this may be done via any method that will permit the location of the tumor and the hardened bone cement to be determined, such as by a CT scan, an MRI or PET scan, etc. Step 1102 may further include step 1202, which may entail transposing the location of the tumor and the hardened bone cement into a coordinate system that is relative to a portion of the body of the patient or other acceptable reference point. The accuracy of the coordinate system an the location of the tumor and other references point(s) is such that it will permit the photon beams to be accurately and safely targeted on the tumor and/or the hardened bone cement or other part of the body. The accuracy is also such that it will permit a radiotherapy regime to be developed that will purposely rely on the Compton interaction knock-out electrons and will permit the influence of those electrons on the tumor to be estimated. In an exemplary embodiment, step 1102 includes step 1203, which entails constructing a model, such as a computer model, of the location of the tumor and the hardened bone cement in the body of the patient. This model may include other features of the patient, such as the location of the spinal cord or other body parts, the location of which is important to know for the procedure. At step 1204, photon beam directions relative to the body of the patient and/or relative to other features (e.g., the tumor and/or the hardened bone cement) are established for each photon beam that will be applied in step 1103. In establishing these directions, the effects of the Compton interaction knock-out electrons are accounted for/the directions are established so that the effects of the Compton interaction knock-out electros are accounted for and/or taken advantage of as detailed herein.

In step 1204, at least one of a total one beam radiation dose and a total collective radiation dose applied to the tumor and/or other portions of the patient's body are determined for the directions of the beams (based on the energy of the beams). This determination of the total one beam radiation dose includes taking into account the effect of the Compton interaction electrons knocked-out from the hardened bone cement as a result of the photon beam(s) impinging upon the hardened bone cement. In an exemplary embodiment, step 1103 may include specifically estimating the secondary one beam radiation dose for one or more of the beam directions, or the secondary collective radiation dose for all of the beam directions. Step 1204 may rely on the model constructed in step 1203.

In step 1204, for at least one photon beam, the direction of the photon beam is established based on the effects of the Compton interaction knock-out electron(s) impinging on the tumor. In an embodiment, this includes estimating the total one beam radiation dose, the total collective radiation dose, the secondary one beam radiation dose and/or the secondary collective radiation dose, where either of the total radiation doses include the dose resulting from the Compton interaction knock-out electrons (the secondary radiation dose).

Accordingly, the radiation regime which is developed in step 1102 purposely takes into account the effects of the Compton interaction knock-out electron(s).

Step 1204 may include the action of loading information regarding the beam directions in to a computer 777 so that the photon beams may be automatically directed at their targets during application of the radiotherapy regime of step 1103. In any event, in step 1103, the photon beams are directed at their targets based on the directions determined in step 1102, which were determined based in part on the effects of the Compton interaction knock-out electron(s), as detailed above. In an exemplary embodiment, one or more of the above method steps may, in whole or in part, be practiced on an electronic computer or a series of electronic computers.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A method of treating diseased tissue in a patient, the diseased tissue being proximate a hardened previously implanted bone cement including relatively high atomic number elements in a patient, comprising:
generating a photon beam; and
directing the generated photon beam into the patient in a direction such that at least a portion of the photon beam impinges on the hardened bone cement and generates Compton interaction knock-out electrons from the high atomic number elements included in the hardened bone cement as a result of interaction of the at least a portion of the photon beam with the bone cement,
wherein the direction of the photon beam is such that the at least a portion of the photon beam impinges on the hardened bone cement so that at least some of the Compton interaction knock-out electrons impinge upon the diseased tissue.

2. The method of claim 1, wherein the method is a method of treating a spinal metastasis in a patient, wherein the spinal metastasis includes the diseased tissue, wherein the diseased tissue is a tumor.

3. The method of claim 1, wherein the relatively high atomic number is about 65-80.

4. The method of claim 1, wherein the relatively high atomic number elements includes tantalum that provides a source for the Compton interaction knock-out electrons.

5. The method of claim 4, wherein the tantalum comprises about 20% by weight of the hardened cement.

6. The method of claim 4, wherein the tantalum comprises about 40% by weight of the hardened cement.

7. The method of claim 2, further comprising:
directing the photon beam into the patient in a direction such that the photon beam impinges on the tumor, thereby providing a primary radiation dose to the tumor,
wherein a secondary ration dose provided to the tumor by the Compton interaction knock-out electrons is about 5% or more of the primary radiation dose.

8. The method of claim 7, wherein the wherein a secondary ration dose provided to the tumor by the Compton interaction knock-out electrons is about 10% or more of the primary radiation dose.

9. The method of claim 1, further comprising shielding non-diseased tissue proximate the bone cement from at least a portion of the photon beam.

10. The method of claim 1, wherein a radiation dose received by tissue at a location aligned with the direction of travel of the photon beam but on an opposite side of the bone cement from the location at which the photon beam impinges on the hardened cement is lower than the radiation dose that would have been received at that location in the absence of the bone cement.

11. The method of claim 1, wherein the hardened previously implanted bone cement is located in a decompressed vertebral body.

12. The method of claim 1, wherein:
the generated photon beam is directed into the patient in a direction such that the photon beam impinges upon the diseased tissue.

13. The method of claim 1, wherein:
the generated photon beam is directed into the patient in a direction such that at least a portion of the photon beam impinges upon the diseased tissue prior to the at least a portion of the photon beam that impinges upon the hardened bone cement impinging upon the hardened bone cement.

14. The method of claim 13, wherein:
the at least a portion of the generated photon beam that impinges upon the diseased tissue delivers a primary one beam radiation dose to the tumor;
at least a portion of the at least a portion of the generated photon beam that impinges upon the diseased tissue continues past the diseased tissue to impinge upon the hardened bone cement; and
the at least a portion of the generated photon beam that impinges on the hardened bone cement generates the Compton interaction knock-out electrons.

15. A composition for bone cement used in at least one of kyphoplasty and vertebroplasty consisting essentially of:
relatively high atomic number elements at about 20% to 40% by weight prior to hardening of the bone cement; and
polymethylmethacrylate (PMMA).

16. The composition of bone cement of claim 15, wherein:
the relatively high atomic number elements consist essentially of tantalum.

17. The composition of bone cement of claim 15, wherein:
relatively high atomic number elements are at about 40% by weight prior to hardening of the bone cement; and
the relatively high atomic number elements consist essentially of tantalum.

18. A method of treating spinal metastasis in a patient, the spinal metastasis including a tumor proximate a hardened bone cement in a vertebral body of a spinal body of a patient, comprising:
developing a treatment regime for treating the spinal metastasis by:
computationally estimating with an electronic computer a secondary radiation dose to be received by the tumor resulting from Compton interaction knock-out electrons generated from the hardened bone cement as a result of the impingement of at least a portion of the photon beam on the hardened bone cement in a first direction; and
directing a photon beam such that at least a portion of the photon beam impinges on the hardened bone cement to generate the Compton interaction knock-out electrons based on the developed treatment regime so as to provide a secondary radiation dose to the tumor.

19. The method of claim 18, further comprising:
computationally estimating a total one beam radiation dose to be received by the tumor, the total one beam radiation dose including the secondary radiation dose and a primary radiation dose to be received by the tumor resulting from impingement of at least a portion of the photon beam on the tumor.

20. The method of claim 19, further comprising:
directing the photon beam such that at least a portion of the photon beam impinges on the tumor based on the developed treatment regime so as to provide the primary radiation dose to the tumor.
